(19) United States
(12) Patent Application Publication
(10) Pub. No.: US 2015/0314269 A1
(43) Pub. Date: Nov. 5, 2015

(54) PLASMONICALLY ACTIVE NANOCOMPOSITES WITH A BIMODAL NANOPARTICLE SIZE DISTRIBUTION

(71) Applicant: The Research Foundation for the State University of New York, Albany, NY (US)

(72) Inventors: Michael A. CARPENTER, Scotia, NY (US); Zhouying ZHAO, Schenectady, NY (US)

(73) Assignee: The Research Foundation for the State University of New York, Albany, NY (US)

(21) Appl. No.: 14/701,942

(22) Filed: May 1, 2015

Related U.S. Application Data
(60) Provisional application No. 61/987,209, filed on May 1, 2014.

(51) Int. Cl. B01J 23/50; B01J 23/66; B01J 37/08; G01N 33/00; B01J 37/02

(52) U.S. Cl. CPC B01J 23/50; G01N 33/0027; B01J 37/024; B01J 37/08; B01J 23/66

(57) ABSTRACT

A self-regenerative metal nanocomposite comprised of a bimodal distribution of metal nanoparticles (NPs) with a metal oxide surrounding is introduced as a new type of plasmonic catalyst through a physical method. Methods of forming such nanocomposites are also disclosed. The support-free catalyst shows plentiful surface adsorbed oxygen species along with excellent localized surface plasmon resonance (LSPR) and appreciable photoluminescence (PL). The combination of high activity and durability of this plasmonic catalyst makes it viable for potential energy and cost-effective catalytic applications.

### CROSS-REFERENCE TO RELATED APPLICATIONS

This application claims priority under 35 U.S.C. §119 to U.S. Provisional Application No. 61/987,209, filed May 1, 2014, which is hereby incorporated herein by reference in its entirety.

### GOVERNMENT RIGHTS STATEMENT

This invention was made with U.S. Government support under Proposal #1006399 awarded by National Science Foundation. The U.S. Government has certain rights in the invention.

### TECHNICAL FIELD

The present invention generally relates to a method for preparing bimodal nanocomposite materials which harvest light energy for efficient catalytic oxidation of hydrocarbons, carbon monoxide and other chemicals. The nanocomposite has a self-regenerative bimodal particle size distribution where the small particles are highly reactive centers, and the larger particles, while serving as a reactive center, also harvest the majority of the light to aid the reactions of interest.

### BACKGROUND OF THE INVENTION

Nanocatalysts have found a variety of important applications in industrial and environmental fields. The catalysts are typically made from dispersing noble metal (e.g., Ag, Pd) nanoparticles (NPs) with a high ratio of catalytically active surface area onto micro or porous (metal) oxide supports (so-called heterogeneous or supported catalyst) to increase the total surface area and mitigate the inherent thermal instability of metal nanoparticles. One issue is that the loading of the active component of metal NPs has to be kept low, for instance, 8 wt %, to alleviate the instability, which limits the overall catalytic activity of the system. Some residual surfactants from the wet chemical synthesis process or the supports themselves may partially block the active sites on the NP surfaces, thus reducing their activity.

Eliminating catalysis deactivation due to a change in material phase and particle agglomerations in catalytic thermal processes is an active area of materials optimization for extended applications. In contrast, catalytic materials development has rarely employed physical methods involving a high temperature step to produce bare metallic nanoparticle catalysts because of the inevitable Ostwald-ripening-like particle growth and oxidation experienced by these materials during thermal treatment and catalytic reactions, which typically leads to reduced catalytic activity and eventual deactivation. Given these challenges and the benefits of nanostructured catalysts, the continued development of novel nanostructured catalysts is desirable for a variety of applications.

Nanoparticles of noble metals like Ag and Au possess a unique localized surface plasmon resonance (LSPR), a collective oscillation of conduction band electrons in the electromagnetic field of incident light resulting in a resonant optical absorption. In recent years, enhanced chemical reaction rates or reduced input of thermal energy have been observed over plasmonic catalysts for multiple light-introduced processes, for example, water splitting toward H₂ production, decomposition of organic pollutants, CO oxidation, chemical conversion, and room temperature H₂ dissociation. These works have stimulated increased attention and research interest in the use of plasmonic materials to achieve higher reaction rates and energy efficient catalytic processing, in addition to exploring the potential to utilize renewable solar energy. While the enhanced reaction rates are attributable to different plasmonic effects, limited experiments have been conducted thus far to reveal their direct and detailed correlations to gain an understanding of the full mechanism of the plasmonic driven chemistry. For instance, one discussion needed is on the presence and mechanism for producing hot electrons or charged species over plasmonic particles, which has been inferred as an important plasmonic effect for the rate enhancement mechanism.

Evidence in plasmonic nanoparticle catalysts for demonstrating concurrent plasmonic and photoluminescence properties will provide useful information to aid in the elucidation of these processes. Furthermore, in situ monitoring of the surface species and their catalytic interactions with gaseous or liquid agents in concert with the plasmonic properties of metal NPs will provide insight into the mechanism of these plasmon induced chemical reactions. The success of these studies, however, requires thermally stable and strong plasmonically active catalysts with high Raman activity. Further, producing such metal nanoparticles by conventional physical vapor deposition methods is desirable.

### SUMMARY OF THE INVENTION

Briefly, the present invention satisfies the need for highly active and durable nano structures.

The present invention provides, in a first aspect, a bimodal nanocomposite which includes a) a substrate comprising a barrier material disposed upon its surface; b) at least one first spherical or spherical-like metal nanoparticle with a diameter between 30 nm and 200 nm; and c) at least one second spherical or spherical-like metal nanoparticle with a diameter between 1 nm and 30 nm. The ratio between the average diameter of the at least one first metal nanoparticle and the average diameter of the at least one second metal nanoparticle is between 3:1 and 200:1. Particle size can be measured by a number of techniques. For the purpose of the present disclosure, particle size is determined by SEM. The at least one first metal nanoparticle and at least one second metal nanoparticle are disposed upon the barrier material.

The present invention provides, in a second aspect, a method of making a bimodal nanocomposite described herein. The method includes a) providing a substrate; b) depositing a barrier material on the substrate; c) depositing a film of a plasmonically active material on the barrier material and d) annealing the film.

The present invention provides, in a third aspect, a method of using the bimodal nanocomposite described herein. These uses may include Raman tags, emission gas catalyst materials, catalytic oxidation of chemicals, harsh environment gas sensing, surface enhanced Raman studies of nanocatalysis processes, as biosensors, for “lab on a chip” uses, or for the low temperature growth of carbon nanostructures.

The present invention provides, in a fourth aspect, a chemical gas sensor comprising the bimodal nanocomposite described herein.

The present invention provides, in a fifth aspect, a photocatalyst comprising the bimodal nanocomposite described herein.

The present invention provides, in a sixth aspect, a light harvester comprising the bimodal nanocomposite described herein.

The present invention provides, in a seventh aspect, a light converter comprising the bimodal nanocomposite described herein.

These and other objects, features and advantages of this invention will become apparent from the following detailed description of the various aspects of the invention taken in conjunction with the accompanying drawings.

### BRIEF DESCRIPTION OF THE DRAWINGS

FIG. 1 shows secondary electron microscopy (SEM) images of bimodal Ag/AgOₓ nanoparticle structures made from 20 nm precursor Ag films without (a-c) and with (d-f) high temperature catalytic reaction tests at different SEM magnifications.

FIG. 2 depicts X-ray photospectroscopy (XPS) spectra of O 1 s (a) and Ag 3d (b) of a bimodal Ag/AgOₓ nanoparticle structure along with deconvolution curve fitting plotted as dashed lines.

FIG. 3 depicts UV-vis extinction spectra (a) and fluorescence/Raman spectrum (b) of a bimodal Ag/AgOₓ nanoparticle structure characterized at room temperature and ambient air conditions. The dashed line in (b) is for guiding the eye.

FIG. 4 shows Raman spectrum of a bimodal Ag/AgOₓ nanoparticle structure characterized at room temperature and ambient air conditions.

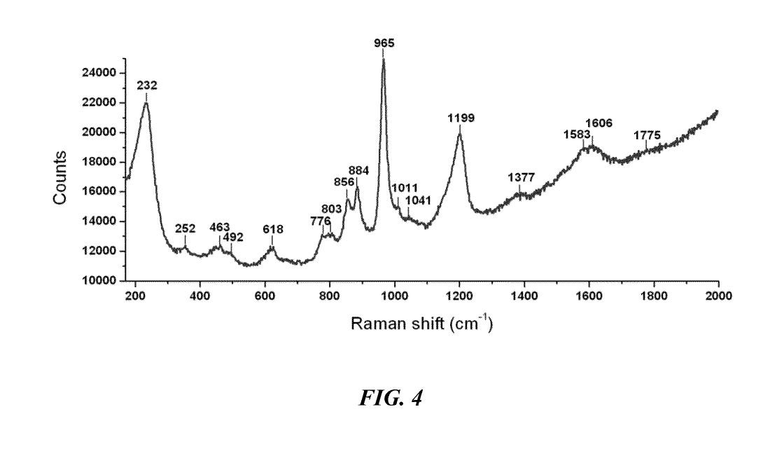

FIG. 5 depicts In situ Raman spectra (a), integrated Raman peak area (b), and extinction spectra (c) of a bimodal Ag/AgOₓ nanoparticle structure as a function of O₂ concentration at 350° C. test conditions. The inset in (c) is the zoomed-in LSPR spectra.

FIG. 6 depicts In situ Raman spectral responses of a bimodal Ag/AgOₓ nanoparticle structure as a function of gas exposure time at room temperature for gases (a) Ar and (b) CO in Ar, where the spectra were collected in ˜10 and 20 min for (a) and (b), respectively. For (b) only one spectrum was taken for each of the CO concentrations, which is increased from 0, 0.5, 0.75, 1 to 1.25%.

FIG. 7 illustrates a reaction that occurs with a composition of the invention.

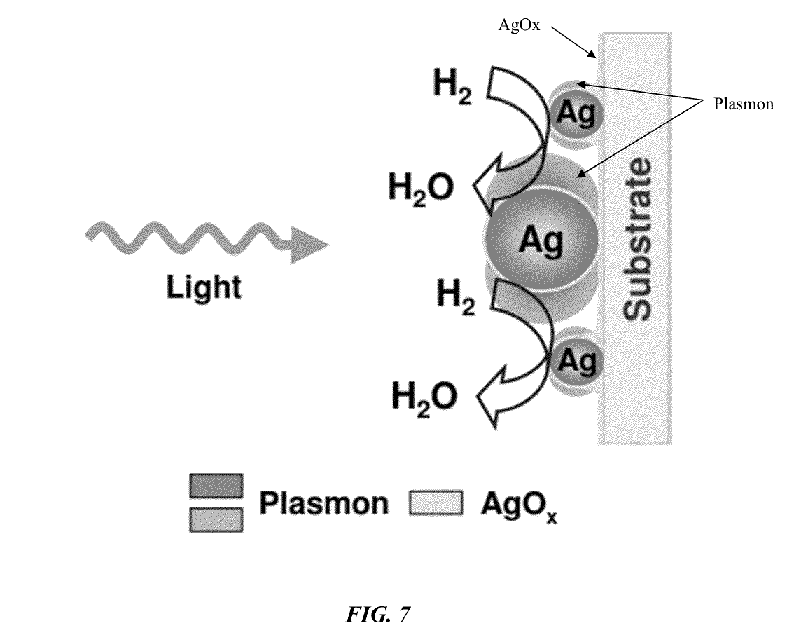

FIG. 8 depicts Scanning electron microscopy (SEM) images of a bimodal Ag/AgOₓ nanoparticle structure made from a 60 nm precursor Ag film at increased SEM magnifications (a, b, c).

FIG. 9 shows X-ray-diffraction (XRD) patterns of a 20 nm precursor Ag film before and after annealing to make the bimodal Ag/AgOₓ nanoparticle structure.

FIG. 10 depicts Secondary ion mass spectroscopy (SIMS) spectrum of a bimodal Ag/AgOₓ nanoparticle structure.

FIG. 11 shows UV-vis spectra of bimodal Ag/AgOₓ nanoparticle structures made from Ag precursor films of different thickness (a) 3 nm and (b) 60 nm, characterized at room temperature and ambient air conditions.

FIG. 12 shows LSPR spectral responses to chemical gas exposures (a and b) and e-SEM images (c and d) for an embodiment of the invention.

FIG. 13 shows a comparison of the sensing traces of LSPR band maxima as a function of H₂ and air exposure time at different temperatures for an embodiment of the invention.

FIG. 14 depicts an LSPR spectral evolution for the embodiment of FIG. 13.

FIG. 15 is a drawing representing a hypothetical explanation of the observed data.

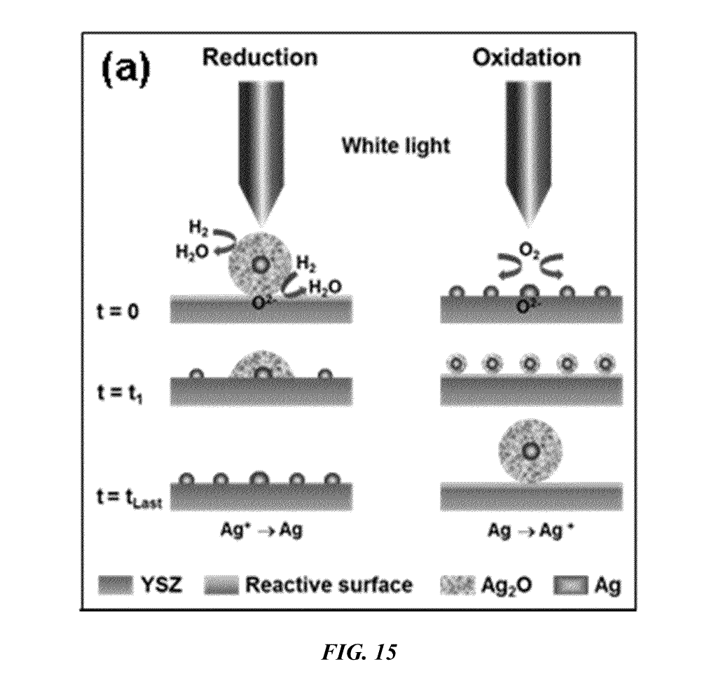

### DETAILED DESCRIPTION OF THE INVENTION

Aspects of the present invention and certain features, advantages, and details thereof, are explained more fully below, and references are made to the non-limiting embodiments illustrated in the accompanying drawings (which are not necessarily drawn to scale). Descriptions of well-known materials, fabrication tools, processing techniques, etc., are omitted so as to not unnecessarily obscure the invention in detail. It should be understood, however, that the detailed description and the specific examples, while indicating embodiments of the invention, are given by way of illustration only, and are not by way of limitation. Various substitutions, modifications, additions and/or arrangements within the spirit and/or scope of the underlying inventive concepts will be apparent to those skilled in the art from this disclosure.

Plasmonically active materials often utilize metal nanoparticles. The creation of a nanocomposite with a bimodal nanoparticle size distribution using physical vapor deposition and thermal processing involves the use of a thermodynamic barrier towards the growth of the nanoparticles. The barrier used for hindered particle growth is deposited with a decreasing concentration with increased film thickness. This barrier material should preferentially oxidize before oxidation of the plasmonically active material, thus enabling the hindered growth of the nanoparticles.

The model for this process can be summarized for the specific case of a Ag/AgOx nanocomposite formed using an Al/AlOx barrier, although other materials described herein may be used. Aluminum is deposited on a substrate in trace quantities with a fractional gradient that decreases with increasing film thickness of the plasmonically active material. For the case of a Ag/AgOx nanocomposite, a Ag film is deposited as the plasmonically active material. Upon annealing, Ag beyond the bottom portion of the film, in the presence of less trace Al, can readily grow into larger Ag particles via an Ostwald ripening model along with metal oxidation. Ag closer to the bottom of the film is within the distribution of more trace Al, which is preferentially oxidized and produces a thermodynamic barrier towards the growth of AgNPs. This leads to the formation of a uniform distribution of smaller sized AgNPs dispersed in an AlOx (trace)-AgOx “blanket”.

This method is unique in that it allows for the preparation of plasmonically active nanocomposites with a bimodal distribution of metal nanoparticles. These nanoparticles are active participants in catalysis, chemical sensing, Raman activity, fuel cell technologies, and solar energy harvesting applications at ambient and elevated temperatures. As they are prepared using only physical vapor deposition and thermal processing methodologies, there are no surfactant agents or metal oxide support materials which block the activity of the nanoparticles. Thus nanocomposite materials deposited and processed in this fashion show higher chemical activity and higher thermal stability than those produced from a wet chemistry or sol gel materials process.

As used herein, the following definitions shall apply unless otherwise indicated. For purposes of this invention, the chemical elements are identified in accordance with the Periodic Table of the Elements, CAS version, Handbook of Chemistry and Physics, 75th Ed.

The present invention provides, in a first aspect, a bimodal nanocomposite which includes a) a substrate comprising a barrier material disposed upon its surface; b) at least one first spherical or spherical-like metal nanoparticle with a diameter between 30 nm and 200 nm; and c) at least one second spherical or spherical-like metal nanoparticle with a diameter between 1 nm and 30 nm. The ratio between the average diameter of the at least one first metal nanoparticle and the average diameter of the at least one second metal nanoparticle is between 3:1 and 200:1. The at least one first metal nanoparticle and at least one second metal nanoparticle are disposed upon the barrier material.

The nanocomposite includes a substrate. In some embodiments, the substrate may be an inert material. In some embodiments, the substrate is quartz. In other embodiments, the substrate is glass. In still other embodiments, the substrate is sapphire. In yet other embodiments, the substrate is silicon.

The substrate has a barrier material disposed upon its surface. The barrier material may be any material that is more easily oxidized than the plasmonically active material during subsequent annealing. In some embodiments, the barrier material is aluminum. In other embodiments, the barrier material is titanium. In still other embodiments, the barrier material is zinc. In yet other embodiments, the barrier material is zirconium.

In some embodiments, the substrate may include a coating on its surface, such as a metal oxide or other catalytically and/or functionally active material. The inclusion of the coating enhances the catalytic properties of the subsequently-formed nanoparticles. In some embodiments, the metal oxide is yttria stabilized zirconium oxide. In other embodiments, the metal oxide is titanium dioxide. In still other embodiments, the metal oxide is cerium dioxide. In those embodiments in which a coating is present on the surface of the substrate, the barrier material will be present on the coated surface of the substrate. To be perfectly clear, the term “substrate” as used herein may refer to a coated or uncoated substrate.

The at least one first metal nanoparticle and at least one second metal nanoparticle (described more fully below) are disposed upon the barrier material.

For purposes of this disclosure, “disposed upon” means that the nanoparticles, after their formation, are located on the topmost surface of the barrier layer, that is, the surface furthest from the substrate. In some embodiments, one or more additional inert layers may be present between the barrier layer and the nanoparticles. In these instances, the nanoparticles may be disposed upon the surface of the layer furthest from the substrate.

The nanocomposite includes at least one first spherical or spherical-like metal nanoparticle. In some embodiments, the diameter of the first nanoparticle is between 30 nm and 200 nm. In some embodiments, the diameter is between 30 nm and 100 nm. In other embodiments, the diameter is between 100 nm and 170 nm. In still other embodiments, the diameter of the first nanoparticle is between 125 nm and 200 nm. In some embodiments, the diameter of the first nanoparticle is between 125 nm and 175 nm. In other embodiments, the diameter of the first nanoparticle is between 75 nm and 125 nm. In some embodiments, the diameter of the first nanoparticle is between 150 nm and 200 nm. In other embodiments, the diameter of the first nanoparticle is between 130 nm and 190 nm. It is to be understood that, if more than one first nanoparticle is present, the average diameter of all first nanoparticles will fall between these described ranges.

The nanocomposite further includes at least one second spherical or spherical-like metal nanoparticle. In some embodiments, the diameter of the second nanoparticle is between 1 nm and 30 nm. In some embodiments, the diameter is between 1 nm and 10 nm. In some embodiments, the diameter is between 3 nm and 8 nm. In other embodiments, the diameter is between 1 nm and 20 nm. In some embodiments, the diameter is between 10 nm and 20 nm. In some embodiments, the diameter is between 5 nm and 15 nm. In still other embodiments, the diameter is between 5 nm and 10 nm. In some embodiments, the diameter is between 2 nm and 8 nm. It is to be understood that, if more than one second nanoparticle is present, the average diameter of all second nanoparticles will fall between these described ranges.

The particle size of all particles present on the surface of the barrier layer, considered in the aggregate, will fall into a bimodal distribution wherein one maximum is at least three times the other. In some embodiments, the ratio between the average diameter of the at least one first metal nanoparticle and the average diameter of the at least one second metal nanoparticle is between 3:1 and 200:1. In some embodiments, the ratio between the average diameter of the at least one first metal nanoparticle and the average diameter of the at least one second metal nanoparticle is between 10:1 and 50:1. In some embodiments, the ratio between the average diameter of the at least one first metal nanoparticle and the average diameter of the at least one second metal nanoparticle is between 20:1 and 100:1. In some embodiments, the ratio between the average diameter of the at least one first metal nanoparticle and the average diameter of the at least one second metal nanoparticle is between 3:1 and 30:1. It is to be understood that, when more than one first nanoparticle and/or more than one second nanoparticle are present, the ratio of the average diameter of all first nanoparticles as compared to the average diameter of all second nanoparticles will fall between these described ranges. As a non-limiting example, in one embodiment, the average diameter of the first metal nanoparticles may be 160 nm, while the average diameter of the second metal nanoparticles may be 10 nm. In this instance, the ratio is 160:10, or 16:1. In another non-limiting example, the average diameter of the first metal nanoparticles may be 30 nm, while the average diameter of the second metal nanoparticles may be 3 nm. In this instance, the ratio is 30:3, or 10:1. If the average diameter of the second metal nanoparticles is at the upper limit of 30 nm, then the smallest average diameter of the first metal nanoparticles may be 90 nm, for an allowed ratio of 3:1.

Both the first and second nanoparticles are composed of a metal. In some embodiments, the metal is selected from silver, nickel, copper, palladium, or platinum. In some embodiments, the metal is selected from silver, nickel, or copper. In some embodiments, the metal is silver. In other embodiments, the metal is nickel. In still other embodiments, the metal is copper. In yet other embodiments, the metal is palladium. In some embodiments, the metal is platinum. In some embodiments, alloys and/or mixtures of these plasmonically active materials, for instance, to form bimetallic nanoparticles, including those containing gold, are utilized. In some embodiments, a metal oxide shell is formed around the first and second nanoparticles. The oxygen in the metal oxide shell comes from the surrounding atmosphere. In most embodiments, the metal oxide shell is formed around the entire particle. There may be instances, however, if the mixed metal nanoparticle is not alloyed, when there could be an oxide formed preferentially around the inner metal, and/or around the outer shell metal.

It is important to note that the size and shape of the nanoparticles can enable the user to control the efficacy of the desired reactions. While spherical and spherical-like nanoparticles are described herein, other nanoparticle shapes with controlled geometries, for instance, rods, pyramids, or triangles, may also be used. In these instances, the size measurement would not necessarily be one of “average diameter”; however, these geometries still will provide the desired characteristics of the bimodal spherical and spherical-like nanoparticles described herein, provided that the ratio of the average sizes of the first and second nanoparticles are similar to those described above. As discussed further below, if the size of the first nanoparticle is too large, desired plasmonic properties are not present. Similarly, if the second nanoparticle size is too great, the desired properties related to catalytic activity decrease.

The present invention provides, in a second aspect, a method of making a bimodal nanocomposite described herein. This method includes providing a substrate. In some embodiments, the substrate may be an inert material, such as quartz, glass, sapphire, or silicon. In other embodiments, the substrate may include a coating on its surface, such as a metal oxide. In some embodiments, the coating is yttria stabilized zirconium oxide. In other embodiments, the coating is titanium dioxide. In still other embodiments, the coating is cerium dioxide.

The method further includes depositing a barrier material on the substrate. The barrier material may be any material that is more easily oxidized than the plasmonically active material during subsequent annealing. In some embodiments, the barrier material is aluminum. In other embodiments, the barrier material is titanium. In still other embodiments, the barrier material is zinc. In yet other embodiments, the barrier material is zirconium. In some embodiments, the deposition of the barrier material is accomplished by thermal evaporation.

The method further includes depositing a film of a plasmonically active material on the substrate. In some embodiments, the plasmonically active material is selected from silver, nickel, copper, palladium, or platinum. In some embodiments, the plasmonically active material is selected from silver, nickel, or copper. In some embodiments, the plasmonically active material is silver. In other embodiments, the metal is nickel. In still other embodiments, the metal is copper. In yet other embodiments, the metal is palladium. In some embodiments, the metal is platinum. In some embodiments, alloys and/or mixtures of these plasmonically active materials, including those containing gold, are used. In some embodiments, the film deposition step occurs after the inclusion of the barrier material to the system. In some embodiments, the barrier material and the plasmonically active material are deposited simultaneously, which allows for the barrier material to have a concentration gradient throughout the film, if desired. In some embodiments, the gradient is such that the portion of the film nearest the substrate has the largest amount of barrier material present, while the portion of the film furthest from the substrate contains essentially no barrier material. In some embodiments, the barrier material is aluminum and the initial aluminum concentration at the substrate is 5 atomic % or less. (The aluminum concentration is with respect to the other materials in the plasmonically active material film). In some embodiments, the initial aluminum concentration at the substrate is between 0.01 at % and 10 at %. In other embodiments, the initial aluminum concentration at the substrate is between 0.1 at % and 7.5 at %. In still other embodiments, the initial aluminum concentration at the substrate is between 0.1 at % and 5 at %. In yet other embodiments, the initial aluminum concentration at the substrate is between 0.5 at % and 5 at %. In some embodiments, the initial aluminum concentration at the substrate is between 1 at % and 5 at %. In other embodiments, the initial aluminum concentration at the substrate is between 1 at % and 3 at %. In still other embodiments, the initial aluminum concentration at the substrate is between 2 at % and 8 at %. In some embodiments, the initial aluminum concentration at the substrate is between 3 at % and 6 at %. In other embodiments, the initial aluminum concentration at the substrate is between 2 at % and 5 at %. In yet other embodiments, the initial aluminum concentration at the substrate is between 3 at % and 5 at %.

The thickness of the plasmonically active material film helps to control the type of nanoparticles produced. If the film is thin, a higher portion of oxidized metal particles are produced, while a film that is thick produces a large portion of large non-optically active large particles. In some embodiments, the thickness of the plasmonically active material film is between 10 nm and 100 nm. In some embodiments, the thickness of the plasmonically active material film is between 15 nm and 60 nm. In some embodiments, the thickness of the plasmonically active material film is between 20 nm and 40 nm. In some embodiments, the thickness of the plasmonically active material film is between 15 nm and 40 nm. In some embodiments, the thickness of the plasmonically active material film is between 15 nm and 25 nm. In some embodiments, the thickness of the plasmonically active material film is 20 nm.

The method further includes annealing the substrate, deposited barrier material, and plasmonically active material Annealing parameters are well known in the art and will not be described in detail here. The annealing step should be performed at a time and temperature adequate to allow for the completion of the desired first and second nanoparticle growth; that is, the time and temperature parameters should be such that the first and second nanoparticle sizes are within the desired ranges. In some embodiments, the film is annealed at a temperature between 200° C. and 1100° C. In some embodiments, the film is annealed at a temperature between 600° C. and 1100° C. In some embodiments, the annealing is tailored in a stepwise fashion. The annealing step should take place in a highly pure, non-reactive gas. In some embodiments, the purity of the gas is between 99% and 100%. In some embodiments, the purity of the gas is between 99.99% and 100%. In some embodiments, the purity of the gas is 99.999%. In some embodiments, the highly pure, non-reactive gas is argon, nitrogen, or helium. In some embodiments, the highly pure, non-reactive gas is argon. In some embodiments, the film is annealed at 900° C. in 99.999% Ar of 2000 sccm flow for 10 minutes with a ramp time of 30 minutes.

The bimodal nanocomposite disclosed herein may be used for a number of different applications. They may be used for Raman tags, emission gas catalyst materials, catalytic oxidation of chemicals, harsh environment gas sensing, surface enhanced Raman studies of nanocatalysis processes, as biosensors, for “lab on a chip” uses, or for the low temperature growth of carbon nanostructures.

In one aspect, the invention relates to a highly active and durable nanostructure comprised of a self-regenerative bimodal distribution of surfactant/support-free plasmonic AgNPs with a surface AgOₓ layer, prepared purely with a physical method. It is known that small noble metal particles have high catalytic activity but poor plasmonic activity, which is opposite to that of large particles. Therefore, conventional heterogeneous catalysts utilizing small particles tends to have poor plasmon enhancement benefits if the LSPR is in the UV region and unable to absorb abundant visible light. The bimodal system in the present study combines small metal nanoparticles as highly active catalyst centers and the large particles with strong visible wavelength LSPR as hot spots to plasmonically enhance the activity of their own and likely the small particles in proximity as well, providing an overall higher catalytic activity. Furthermore, the large AgNPs thermodynamically have a higher resistance to oxidation under the catalytic operation conditions and are able to retain a strong LSPR for desired plasmonic enhancement effects over small particles, which are easily oxidized and lose their LSPR strength. The nanostructure contains a trace amount of Al oxide which, with a high temperature thermal equilibration step, enables long-term thermal stability without the use of a high loading of foreign oxide support or cap, thus maximizing the plasmonic activity of the system. Indeed, not only LSPR extinction but also photoluminescence are observed from these samples, the latter of which testifies to the generation of electron-hole pairs from enhanced interband transitions by the plasmon induced local field. Further, plasmon induced preferential growth of surface species is demonstrated over the nanostructure with in situ Raman spectroscopy based studies. The direct correlation between LSPR and surface species/reactions is consistent with results reported in the recent literature based on the measurements of chemical reaction products and is essential for the further development of plasmonically enabled photocatalysis studies. This newly developed Ag/AgOₓ nanostructure can potentially serve as an efficient system for multiple applications including catalytic oxidation of chemicals, harsh environment gas sensing, surface enhanced Raman studies of nanocatalysis processes, and low temperature growth of carbon nanostructures.

Although this invention is susceptible to embodiment in many different forms, certain embodiments of the invention are shown and described. It should be understood, however, that the present disclosure is to be considered as an exemplification of the principles of this invention and is not intended to limit the invention to the embodiments illustrated.

### Examples

The invention will now be illustrated, but not limited, by reference to specific embodiments described in the following examples.

Polished quartz substrates were cleaned in ultrasonic baths of acetone and ethanol for 10 min. Precursor Ag thin films of 20 nm selected thickness were thermally evaporated onto the precleaned quartz substrates from 99.99% pure Ag pellets loaded in a tungsten boat (both from K. J. Lesker) in a high vacuum chamber with a base vacuum of ˜10⁻⁷ Torr. Each of the Ag film depositions was preceded by a separate Al (99.99% pure) thermal evaporation (˜1 μm thick) deposition, which coated the walls of the evaporator with Al. This predeposition was used to intentionally introduce a trace amount of Al into the subsequently deposited Ag film, through re-evaporation of the Al precoated on the chamber components at/near the thermal evaporation source. In this way the Al content in the Ag film is reduced with increased Ag film growth (thickness) as Ag evaporation coats the vacuum chamber during its deposition process. Introduction of this type of gradient trace Al distribution aids in the formation of the bimodal distribution of Ag/AgOₓNP structure via a high temperature process as described below.

After removal from the thermal evaporation chamber, the Ag film was annealed at 900° C. in 99.999% Ar of 2000 sccm flow for 10 min with a ramp time of 30 min followed by a natural cooling in the carrier gas. Prior to the annealing, the sample was pumped down to ˜10 Torr for 10 min and then purged with 2000 sccm Ar for 15 min to minimize residual O₂ levels. Minimizing residual O₂ in the furnace alleviates excessive Ag oxidation at elevated temperatures. The as-prepared Ag/AgOₓ nanostructure sample was then cured in a mini (˜10 cm³ volume) catalytic reaction cell at 400° C. in 10% O₂ for ˜15 h for activation of Raman activity. The cell allows heating to 450° C. in different redox gases (O₂, H₂, and CO balanced with Ar) with a total gas flow of 400 sccm at a pressure of slightly less than 1 atm. A Renishaw Raman spectrometer was coupled to the cell through a Leica optical microscope for in situ Raman studies. A 514.5 nm Ar ion laser was used as the excitation source with the laser power at the sample surface being ˜1.5 mW. An acquisition time of 40 s was used for the Raman data collection. For characterization of catalytic reactions, the sample was loaded in the cell and exposed to 10% O₂ in Ar for 1 h after heating to the targeted operation temperature (e.g., 300° C.) for pretreatment, followed by catalytic reaction data collections in 20-25 min intervals for gas and thermal stabilization in each consecutive reaction using different gases. The Raman spectrum was collected on a thicker film area made with scratching the Ag/AgOₓ film to enhance the Raman signals and to minimize background Raman signals from the quartz substrate, as described further below.

### Results

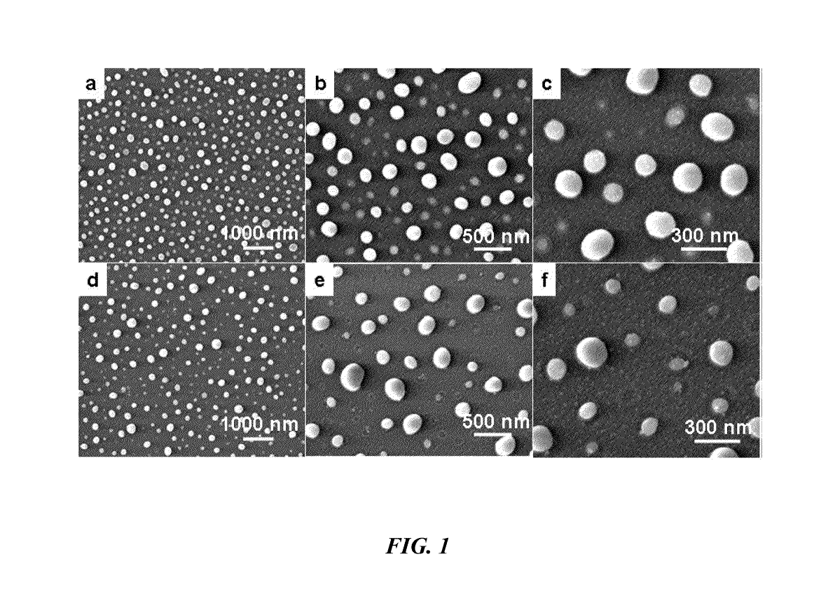

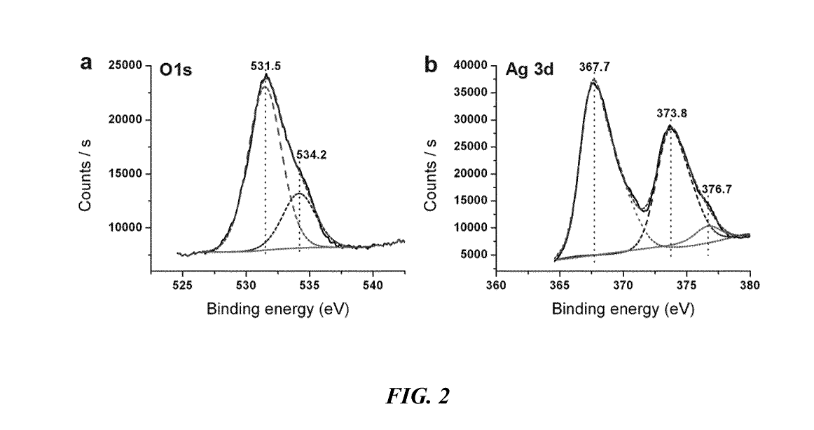

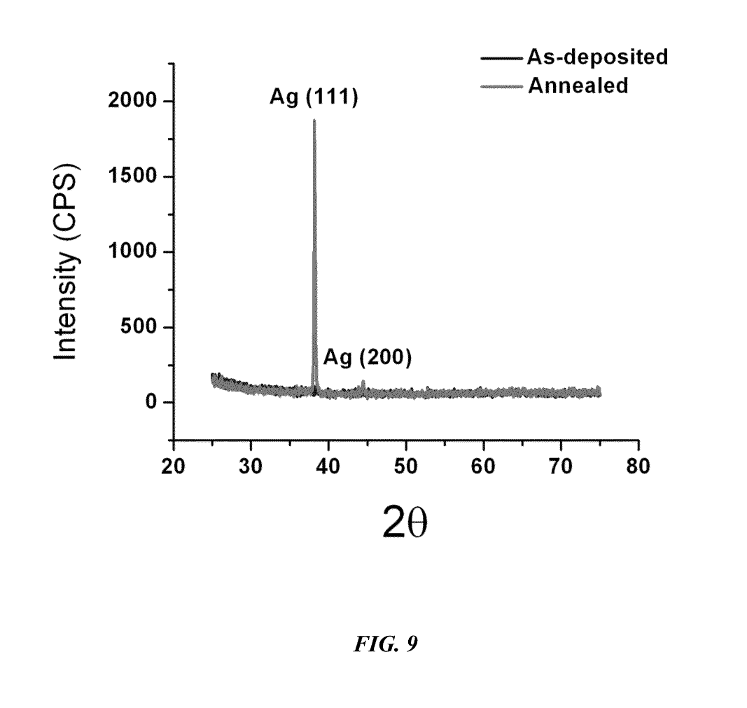

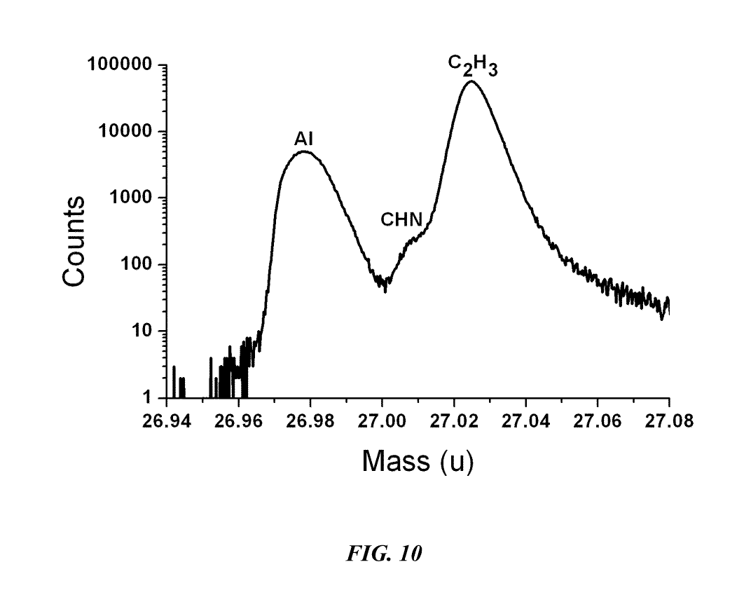

The support-free self-regenerative Ag/AgOₓ nanoparticle structure with plasmon activity was produced using a two step solid phase procedure: thermal evaporation of a precursor Ag thin film on a quartz substrate and then annealing at 900° C. for 10 min in 2000 sccm flow of 99.999% Ar. A unique part of the processing developed in this work is that each of the Ag film depositions is preceded by a separate run of Al thermal evaporation for intentionally introducing a gradient trace quantity of Al into the film. Conventionally, support-free nanoparticle catalysts have received limited attention due to unconstrained particle growth/agglomeration and fast deactivation during thermal processing and catalytic applications. The size and oxidation of the AgNPs formed at high temperature may be modulated by control of the film thickness, Al doping, and annealing ambient conditions. FIG. 1 displays environmental secondary electron microscopy (SEM) images of two separate nanostructures made from 20 nm thick Ag films, without and with catalysis tests at high temperature, respectively. For the as-prepared nanostructure, the images in FIG. 1a-c, with increasing magnification, show a distribution of well-separated large AgNPs (˜160 nm on average size) sitting on a “blanket” comprised of a small uniform size of AgNPs (˜6 nm on average). These small nanoparticles are well dispersed in a mainly AgOₓ surrounding, a thin layer of which is observable on the large AgNPs as well. The chemical contents are confirmed with the composition and optical characterizations shown below. This structure, different from those with one size set of dense or bulk-like large metal NPs reported in the literature, combines the small size of AgNPs required for high catalytic activity and the large AgNPs with strong LSPR intensity for reinforcing catalytic activity while capped by a stabilizing layer of AgOx. The second sample shown in FIG. 1d-f has undergone repeated high temperature (up to 400° C.) gas exposure tests for more than 150 h plus a higher temperature (900° C.) CO exposure for 30 min and has average particle sizes for the large and small particles of ˜190 and 10 nm, respectively. The comparison in FIG. 1, despite not being for exactly the same sample, indicates a general repeatability in forming the support-free catalyst with its bimodal particle size distribution. XRD spectral analysis confirms the formation of crystalline metallic AgNPs with (111) preferred orientation (FIG. 9), where the Ag (111) plane is supposed to be favorable for oxygen surface species formation based on studies using bulklike Ag catalysts. XPS, as a surface sensitive (˜10 nm) analysis technique, reveals Ag2O/AgO phases on the AgNP surface and a AgOₓ phase within the subsurface or bulk of the particles, according to the binding energies of 531.5 and 534.2 eV, respectively, with the deconvoluted O is core level peaks shown in FIG. 2a. The larger XPS peak of Ag 3d in FIG. 2b is shifted to a lower binding energy of 367.7 eV relative to 368.4 eV for metallic Ag 3d due to bonding with oxygen, confirming the formation of the surface AgOx phases. The small shoulder at a binding energy of 376.7 eV may represent Al 2p, in agreement with the trace Al detection by secondary ion mass spectroscopy (SIMS) shown in FIG. 10. The presence of surface AgOₓ is beneficial not only for capping and enabling the self-regeneration of the AgNPs but also for catalytic reactions, as the nature of Ag catalysts for chemical conversions, such as for ethylene epoxidation, has been found to be associated with AgOₓ. The extensive AgOₓ surface area on the bimodal AgNPs in the present nanostructure is thus a significant benefit for surface catalytic chemical reactions as compared to bulk-like catalysts with limited surface area or as compared to supported catalysts with potential surface site blockage.

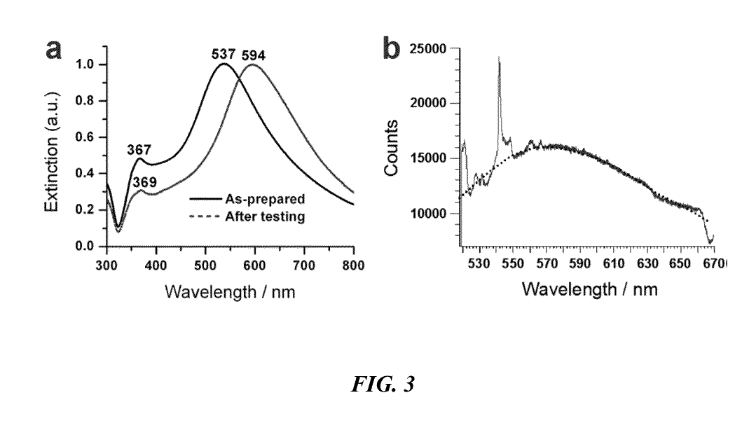

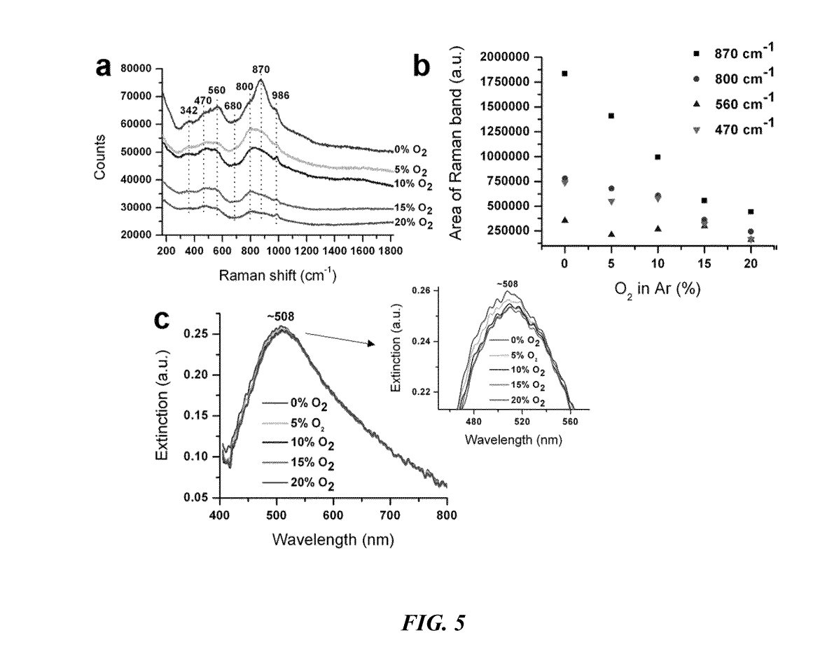

FIG. 3a displays the extinction spectra for the Ag/AgOₓ NP structure before and after gas exposure tests. Both spectra are characterized with distinctly large and small LSPR bands at visible and ultraviolet (UV) wavelength ranges, respectively, as marked in the figure. These peaks in general correlate well with the large and small average sizes of the AgNPs outlined above, confirming the bimodal AgNP morphology. Particle growth due to the high temperature processes appears to be a minimal contributor, as shown in FIG. 5c, where the LSPR maxima of a tested sample remains in the wavelength region similar to that of the as-prepared sample shown in FIG. 3a for nearly 80 h during repeated O₂ exposures, surface “cleaning”, at 350° C., affirming the thermal stability of the nanocomposite system.

LSPR enhanced local electrical fields and particularly surface or interface transfer of energetic electrons from resonant formation of plasmons will facilitate surface species growth, adsorbate interactions, and their corresponding dynamics. Thus, by optimizing plasmonic energy through metal nanoparticle size and shape control, catalysts can be developed with a high catalytic activity via LSPR coupling.

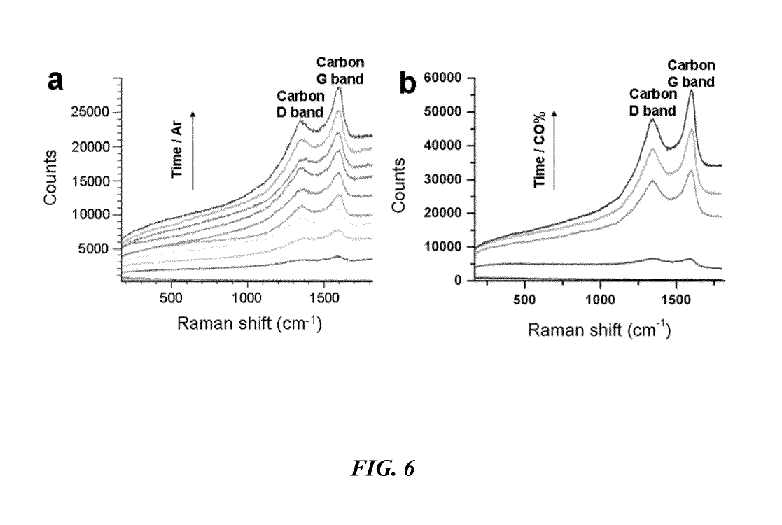

Room temperature activity and regeneration of the catalyst are of particular interest as this renders more energy efficient processing and wider applications. As shown in FIG. 6, upon exposure of the Ag/AgOₓ nanostructure to 400 sccm Ar (99.999% pure), distinctive Raman peaks at 1335 and 1580 cm⁻¹ appear while the Raman peaks for all other surface oxygen species are barely visible (FIG. 6a). These two peaks are characteristic of amorphous carbon with the well-known D and G bands of sp2-hybridized configurations. In contrast, when catalytically active CeO₂ thin films are exposed to this same gas, none of these Raman peaks for carbon are observed (not shown). It appears that the surface oxygen species on the Ag/AgOₓ nanostructure catalytically oxidize trace sources of hydrocarbons from the gas manifold. The fast and complete formation of amorphous carbon on the Ag/AgOₓ NP structure at RT is another example of its high catalytic activity. When the feed gas contains 0-1.25% CO, the D and G bands of carbon peaks increase further with CO concentration and become substantially larger (FIG. 6b) and are as sharp as that for intentionally grown carbon nanostructures. This suggests that CO oxidation takes place at RT, especially over the plasmonic AgNPs with strong LSPR, leading to AgOₓ reduction. This in turn increases the Ag fraction and the associated LSPR in the self-regenerative NP structure, thus enhancing not only surface Raman scattering but also surface species growth and oxidation of adsorbed hydrocarbons as a function of CO concentration. The enhanced Raman signal during CO exposure is consistent with that observed from FIG. 5. In both cases, plasmonically coupled growth of surface species increases when CO is increased or O₂ is reduced, substantiating the coupling of the LSPR with surface catalytic properties as an important feature to enhance catalytic processes even at RT. When exposed to O₂ at RT, the carbon peaks diminish or disappear while surface oxygen Raman signals increase, illustrating the catalyst excellent room temperature activity and self-revitalizing capability.

Given these results, it is expected that when applied to catalytic oxidation of chemicals under industrial conditions, the self-regenerative plasmonic Ag/AgOₓ NP structure can serve as an efficient catalyst while maintaining a high durability at elevated temperatures. It can act as a thermally robust platform for study of catalysis processes or as an effective platform for study of biomolecules with surface enhanced Raman scattering. It should also allow for low temperature catalytic growth of graphitic carbon nanostructures as reported by Hung et al. using a AuNP system.

### Preparation of Ag/AgOx Nanoparticle Structure

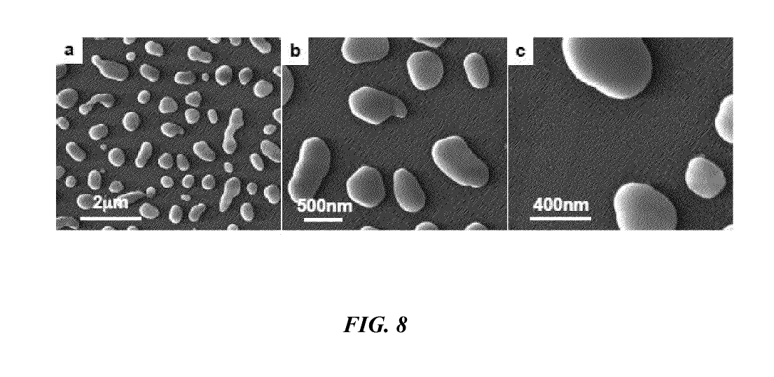

The precursor Ag film thickness plays a significant role on the Ag/AgOx nanostructure formation and composition via the high temperature annealing process outlined in the Methods. For example, a 3 nm thick Ag film is fully oxidized during the annealing cycle and shows no observable LSPR band. For a 60 nm Ag film, although a bimodal distribution of Ag/AgOx NPs results, the large particles have an average size of ˜490 nm, which is too large to produce the desired LSPR in the visible light range. FIG. 8a,b,c displays SEM images of the Ag/AgOx NP structure obtained from a 60 nm Ag film for a comparison with that obtained from a 20 nm Ag film (FIG. 1) at different SEM magnifications. It is noticeable that other than the larger AgNPs, a uniform distribution of small size (on average ˜10 nm) AgNPs is present and comparable to that seen in FIG. 1f, consistent with the formation of a bimodal nanoparticle structure. The bimodal properties of AgNPs are verified by the UV-vis results shown later.

### Characterization

X-ray diffraction (XRD): FIG. 9 shows XRD patterns for a 20 nm thick Ag film before and after annealing. There are no XRD peaks observed with the as-deposited film, while after annealing at 900° C., a sharp peak appears at 2Θ of 38.18°, indicating (111) as the preferred orientation for the formed AgNPs.

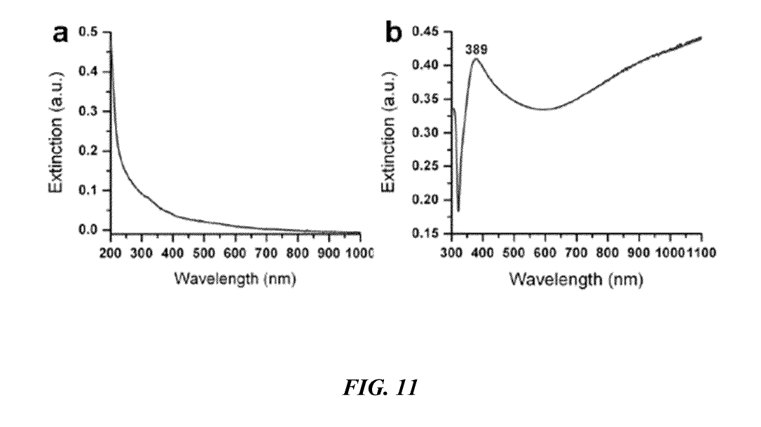

Secondary ion mass spectroscopy (SIMS): As detailed herein, trace Al is introduced into the Ag film via reevaporation of the Al pre-coated on the chamber components during the subsequent Ag thermal evaporation process. Thus the trace Al will be enriched at the bottom of the Ag film according to the physical deposition nature. The presence of Al in the sample is confirmed by the secondary ion mass spectrometry (SIMS) spectrum as shown in FIG. 10. Without being held to any one theory, the following model for the nanostructure growth is proposed. Upon annealing, Ag beyond the bottom portion of the film is in the presence of less trace Al and can readily grow into larger Ag particles via an Ostwald ripening model along with metal oxidation. Ag closer to the bottom of the film is within the distribution of more trace Al, which is preferentially oxidized and produces a thermodynamic barrier towards the growth of AgNPs. This leads to the formation of a uniform distribution of smaller sized AgNPs dispersed in an AlOx (trace)-AgOx “blanket”. This model accounts for the growth of a bimodal distribution of AgNPs within the Ag/AgOx nanostructure, which is clearly visible from the SEM images in FIG. 1 and FIG. 8 and the LSPR spectra in FIG. 3 and FIG. 11. It is interesting to note that some small particles appear parasitic on the large particles and remain present even after repeated exposures to catalytic reaction environments (see FIG. 1c and FIG. 1f). They are likely AgNPs with a capping of AgOx-trace AlOx and have been brought up on the larger particles during the nanostructure growth. This self-consistently supports the role of Al and the growth model for the Ag/AgOx nanostructure.

UV-vis extinction spectroscopy: UV-vis extinction spectra are collected in transmission mode on a Varian Cary 50 UVvis spectrometer. FIG. 11 exhibits UV-vis extinction spectra for the nanostructures made from thermal annealing of 3 and 60 nm precursor Ag films, respectively, for a comparison to FIG. 3 for the Ag/AgOx NP structure obtained from a 20 nm Ag film. It can be seen that there is no LSPR band observable in FIG. 11a, illustrating a complete oxidation of the extremely thin 3 nm Ag film during the annealing process. For the 60 nm Ag film, there is a sharp extinction peak present in FIG. 11b, which is centered at 389 nm. The peak position corresponds well with the small sized (on average ˜10 nm) particles visible from FIG. 8c, verifying that the nature of the observed small size particles is indeed metallic Ag instead of AgOx. This agrees well with the case for the 20 nm Ag based system, reinforcing that the small size (averagely ˜6 nm) particles shown in FIG. 1c are metallic AgNPs contributing to the extinction band at 367 nm shown in FIG. 3. This in turn provides further support to the formation of a bimodal AgNP distribution.

It appears that the precursor Ag film thickness has an effect on the size of the large AgNPs formed and thus the associated LSPR. However, Ag film thickness has less of an effect on the small particles and their corresponding LSPR. The small particles appear to be more dependent on the presence of trace Al. In situ Raman spectroscopy: Examination of surface species and their catalytic properties within the bimodal Ag core/AgOx shell nanostructure is carried out through in situ Raman spectroscopy. Table S1 summarizes all of the Raman peaks exhibited in a bimodal Ag/AgOₓ nanoparticle structure characterized at room temperature and ambient air conditions and their potential assignments as noted from previous studies.

TABLE S1
Vibrational Frequency (cm⁻¹) and Assignment of Various
Chemisorbed Oxygen Species Detected in This Study

| Raman shift (cm⁻¹) | species | assignment | refrences |
|---|---|---|---|
| 232 | Ag—O₂⁻¹ | ν(Ag—O₂) | 1, 2 |
| 352 | Ag—O_{γ} | o(Ag—O) | 1, 2 |
| 463 | Ag—O_{α} | ν (Ag—O) | 1, 3 |
| 492 | Ag—OH | ν (Ag—OH) | 1, 4 |
| 618 | Ag—O_{β} | ν (Ag—O) | 1, 2, 5 |
| 776 | Ag [symbol] ═O | ν (Ag—O) | 6 |
| 803 | Ag—O_{γ} | ν (Ag—O) | 1, 2 |
| 855 | Ag—O—Ag | | 7 |
| 883 | Ag—O_{γ}⁻¹ | ν (Ag—O) | 8, 9 |
| 965 | Ag—O₂⁻¹ | ν (O—O) | 1, 2 |
| 1011 | Ag[O—O]⁻¹ | | 10 |
| 1041 | Ag⁻O₂⁻¹ | | 11 |
| 1199 | Ag—O₂⁻¹ | ν (Ag—O₃) + ν (O—O) | 1 |
| 1376 | CO₂ | ν₃₅(CO₃) | 1, 12 |
| 1582 | C related | | 12 |
| 1605 | Ag—O_{γ} | 2ν (Ag—O) | |
| 1775 | ?\| | | |

[symbol]

indicates data missing or illegible when filed

In summary, a support-free self-regenerative nanostructured catalyst was established which contains a bimodal distribution of plasmonic AgNPs with surface AgOₓ. The nanostructure demonstrates a wealth of neutral and ionic molecular and atomic oxygen species on the AgNP surface in addition to strong LSPR and appreciable PL emission signatures. Highly catalytic and durable interactions of surface active atomic oxygen species like Ag—O_{γ} with a Raman peak at ˜800 cm⁻¹ with O₂, H₂, CO, and hydrocarbons at ambient to 300-400° C. temperatures are observed using in situ Raman spectroscopy. The superior surface activity of the nanostructure arises from two factors: the high ratio of unblocked active surface area of the support-free bimodal AgNPs and the strong LSPR character of the AgNPs to further enhance the surface catalytic processes. The durability is achieved for the self-regenerative Ag/AgOₓ nanostructure through incorporation of trace amounts of AlOx and thermal equilibration during a 900° C. annealing cycle. The Ag/AgOₓ NP structure can serve as an efficient catalyst for multiple applications. LSPR enhanced surface species growth and catalytic reactions are directly observed, which is expected to provide valuable information for catalyst and process development.

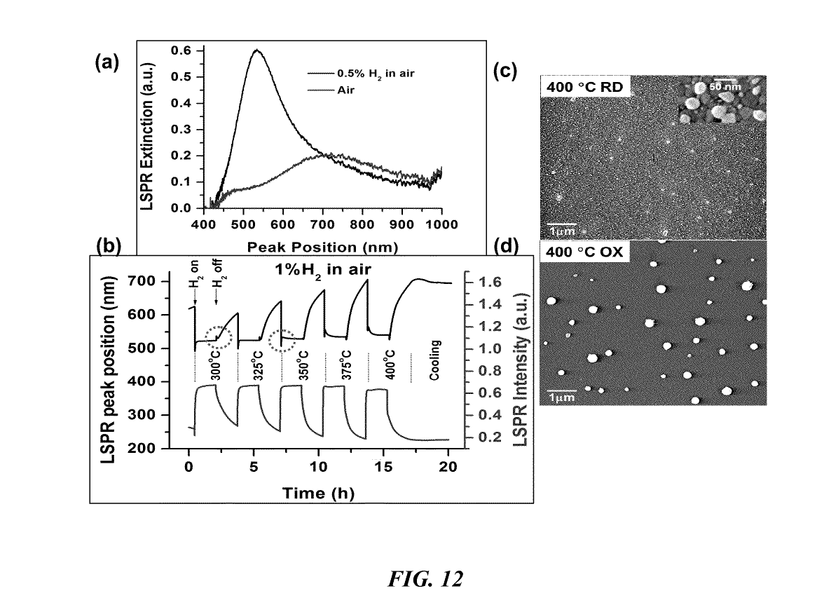

Another example shows that when an ionically active metal oxide support, the well known ionic conductor, yttria stabilized zirconia (YSZ), was functionally coupled to plasmonic Ag nanoparticles (NPs), the material and optical properties under redox conditions are different from that of AgNPs on quartz. Specifically, upon changing from reduction to oxidation conditions, a reversible 200 nm plasmonic response was observed, which coincides with a particle phase/size/morphology oscillation for the Ag/AgOx on YSZ. A stepwise annealing process improves the particle growth on YSZ from the deposited Ag film with a gradient trace Al forming a thermodynamic metal oxide barrier layer that inhibits particle growth. Bimodal AgNPs are consistently obtained for the as-prepared samples as confirmed by two sharp LSPR bands and the SEM images from the as-prepared samples. Gas sensing tests of the sample show large reversible LSPR spectral responses to chemical gas exposures as representatively shown in FIG. 12 a-b for gas cycling between H₂ in air and pure air at temperatures of 300 to 400° C. The LSPR band shift is close to ˜200 nm at 400° C., almost two orders of magnitude larger than that with Au/YSZ nanocomposites and any other plasmonic materials based sensing reported to date. Accompanying the spectral response is a reversible morphology switch between reduced and oxidized sample states with the correspondent small metallic (˜30 nm) Ag particles and large (˜100 nm) Ag core/AgOₓ shell particles oscillating between the two states, as revealed by the e-SEM images in FIG. 12 c-d.

In summary, plasmonically, catalytic and ionically active layered nanocomposites prepared by this novel processing method demonstrate unprecedented and exceptionally large LSPR spectral changes and counter-intuitive microstructure/morphology oscillations involving catalytic redox, plasmonic and ionic effects, and synergistic interactions.

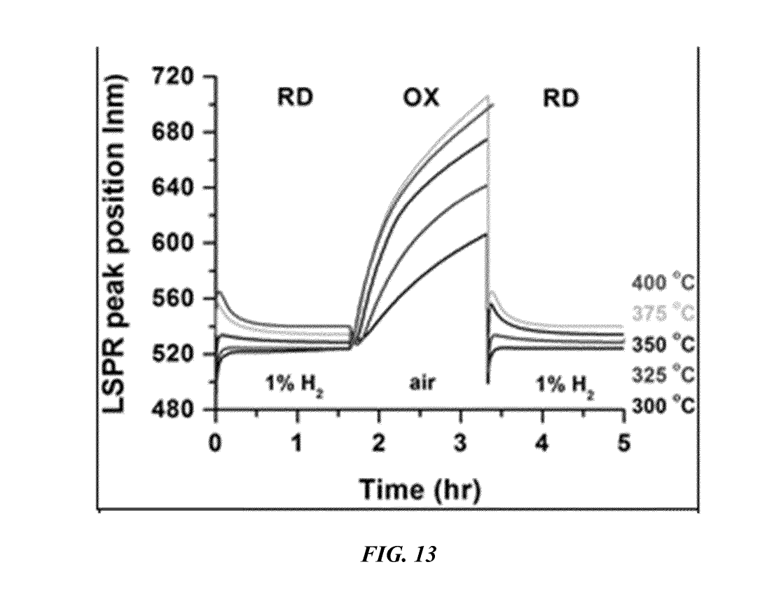

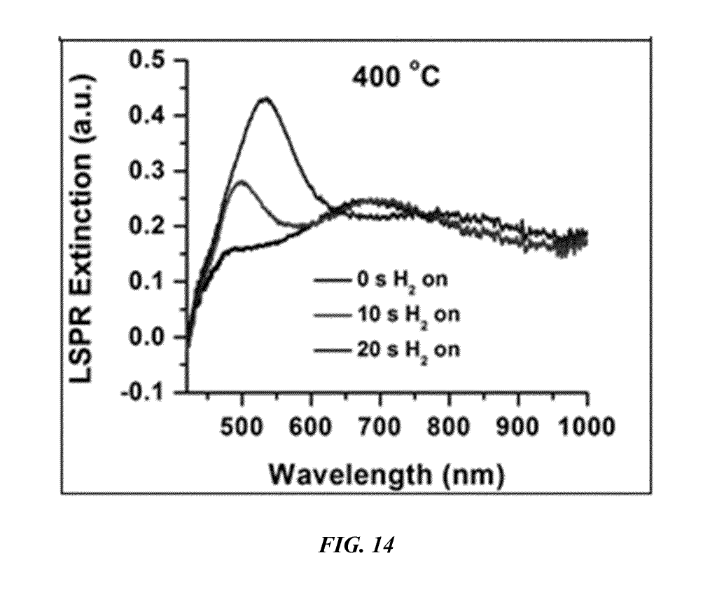

Shown in FIG. 13 is a comparison of the sensing traces of LSPR band maxima as a function of H₂ and air exposure time at different temperatures. Interestingly, the fine LSPR features at the onset of the 1% ΔO₂ gas exposure changes from a spike to a dip shape with temperatures increasing from 300 to 400° C. while only spikes are seen with increasing temperature for the onset of the 1% H₂ exposures. From the correspondent LSPR spectral evolution displayed in FIG. 14, it is clear that these spikes and dips are real, the observation of which is enabled by the highly time resolved LSPR spectra collected (5 to 10 s interval). These fine features vary with temperature and also gas concentration, and should mirror surface/interface chemical reactions and the generated surface species like OH⁻, as LSPR is sensitive to changes in the effective dielectric constant. These spectra are in agreement with previous work showing plasmonically enhanced reactive species resolved by Raman spectroscopy. The Ag/AgOₓ NPs/YSZ structure introduced here acts as a novel platform for study of LSPR coupled surface species and the elementary catalytic steps dictating the dynamic reaction processes on the particle surface/interface.

As demonstrated above, introducing the YSZ layer between the Ag/AgOₓ particles and the quartz substrate improves the LSPR gas response properties.

In summary, silver nanoparticles (AgNPs) were deposited on oxygen ion conducting metal oxide films, which increased their usability as a functional material. In the presence of oxygen and elevated temperatures, 100-500° C., the AgNPs undergo significant growth and mass transfer, which produces large (200 nm-400 nm) core shell particles comprised of a silver oxide shell and a silver metal core. The oxygen exposure can also cause the original bimodal spherical or spherical-like NPs to result in core shell particles of different shapes, including, but not limited to, squares, rods, or ribbons. Upon switching the gas environment to a hydrogen-oxygen mixture the particles again undergo mass transfer and reverse themselves back to the original small silver nanoparticle distribution.

Any numerical values recited in ranges herein include all values between and including the lower value and the upper value.

While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

1. A bimodal nanocomposite, comprising:
a) a substrate comprising a barrier material disposed upon its surface;
b) at least one first spherical or spherical-like metal nanoparticle with a diameter between 30 nm and 200 nm; and
c) at least one second spherical or spherical-like metal nanoparticle with a diameter between 1 nm and 30 nm;
wherein the ratio between the average diameter of the at least one first metal nanoparticle and the average diameter of the at least one second metal nanoparticle is between 3:1 and 200:1; and
wherein said at least one first metal nanoparticle and at least one second metal nanoparticle are disposed upon the barrier material.

2. The bimodal nanocomposite according to claim 1, wherein said metal of said first and second metal nanoparticles is selected from the group consisting of silver, nickel, copper, palladium, and platinum.

3. The bimodal nanocomposite according to claim 2, wherein said metal of said first and second metal nanoparticles is silver.

4. The bimodal nanocomposite according to claim 1, wherein said at least one first spherical or spherical-like metal nanoparticle has a diameter between 130 nm and 190 nm.

5. The bimodal nanocomposite according to claim 1, wherein said at least one second spherical or spherical-like metal nanoparticle has a diameter between 2 nm and 8 nm.

6. The bimodal nanocomposite according to claim 1, wherein the ratio between the average diameter of the at least one first metal nanoparticle and the average diameter of the at least one second metal nanoparticle is between 10:1 and 50:1.

7. The bimodal nanocomposite according to claim 1, further comprising a metal oxide shell on the outer surface of said at least one first metal nanoparticle and said at least one second metal nanoparticle.

8. The bimodal nanocomposite according to claim 7, wherein the metal oxide shell comprises silver oxide.

9. A method of making a bimodal nanocomposite, comprising:
a) providing a substrate;
b) depositing a barrier material on said substrate;
c) depositing a film of a plasmonically active material on said barrier material; and
d) annealing said film;
wherein said bimodal nanocomposite comprises
a) the substrate comprising the barrier material disposed upon its surface;
b) at least one first spherical or spherical-like metal nanoparticle with a diameter between 30 nm and 200 nm; and
c) at least one second spherical or spherical-like metal nanoparticle with a diameter between 1 nm and 30 nm;
wherein the ratio between the average diameter of the at least one first metal nanoparticle and the average diameter of the at least one second metal nanoparticle is between 3:1 and 200:1; and
wherein said at least one first metal nanoparticle and at least one second metal nanoparticle are disposed upon the barrier material.

10. The method according to claim 9, further comprising depositing a coating layer on the substrate prior to the depositing of the barrier layer, wherein said coating layer is a metal oxide.

11. The method according to claim 10, wherein said coating layer is selected from the group consisting of yttria stabilized zirconium oxide, titanium dioxide, and cerium dioxide.

12. The method according to claim 9, wherein said barrier material is selected from the group consisting of aluminum, titanium, zinc, and zirconium.

13. The method according to claim 12, wherein said barrier material is aluminum.

14. The method according to claim 9, wherein said plasmonically active material is selected from the group consisting of silver, nickel, copper, palladium, and platinum.

15. The method according to claim 14, wherein said plasmonically active material is silver.

16. The method according to claim 9, wherein said barrier material is deposited before the deposition of said plasmonically active material.

17. The method according to claim 9, wherein said barrier material and said plasmonically active material are deposited simultaneously.

18. The method according to claim 9, wherein said barrier material has a concentration gradient, wherein the highest concentration of said barrier material is nearest the substrate, and the lowest concentration of said barrier material is furthest from the substrate.

19. The method according to claim 9, wherein said film of plasmonically active material is deposited at a thickness of between 15 nm and 40 nm.

20. A chemical gas sensor comprising a bimodal nanocomposite of claim 1.
